(19) United States
(12) Patent Application Publication
(10) Pub. No.: US 2008/0058418 A1
(43) Pub. Date: Mar. 6, 2008

(54) STABLE POLYUNSATURATED FATTY ACID EMULSIONS AND METHODS FOR INHIBITING, SUPPRESSING, OR REDUCING DEGRADATION OF POLYUNSATURATED FATTY ACIDS IN AN EMULSION

(75) Inventors: Lihong D'Angelo, Decatur, GA (US); Youlung Chen, Marietta, GA (US); George A. King, Atlanta, GA (US)

Correspondence Address: SUTHERLAND ASBILL & BRENNAN LLP, 999 PEACHTREE STREET, N.E., ATLANTA, GA, 30309, US

(73) Assignee: THE COCA-COLA COMPANY, Atlanta, GA (US)

(21) Appl. No.: 11/850,158

(22) Filed: Sep. 5, 2007

Related U.S. Application Data
(60) Provisional application No. 60/824,709, filed on Sep. 6, 2006.
(60) Provisional application No. 60/888,256, filed on Feb. 5, 2007.
(60) Provisional application No. 60/948,338, filed on Jul. 6, 2007.

(51) Int. Cl. A61K 31/20; A61K 31/047; B01F 3/08; C07C 51/50

(52) U.S. Cl. 514/560; 514/729; 516/9; 554/2; 554/7

(57) ABSTRACT

An emulsion comprising a continuous liquid phase, an emulsifier, and a discontinuous liquid phase comprising a blend including a polyunsaturated fatty acid source and a dispersing agent. The polyunsaturated fatty acid source comprises at least one polyunsaturated fatty acid and the weight ratio of the fatty acid source to the dispersing agent in the blend ranges from about between 9:1 and 1:10. A method for making an emulsion. The stability of the emulsion can be protected by antioxidants such as tea polyphenols.

### CROSS-REFERENCE TO RELATED APPLICATIONS

This application claims priority under 35 U.S.C. §119(e) to U.S. Provisional Patent Application No. 60/824,709, filed on Sep. 6, 2006, U.S. Provisional Patent Application No. 60/888,256, filed on Feb. 5, 2007, and U.S. Provisional Patent Application No. 60/948,338, filed on Jul. 6, 2007, the disclosures of which are expressly incorporated herein by reference.

### TECHNICAL FIELD

This invention relates to methods for inhibiting, suppressing, or reducing degradation of polyunsaturated fatty acids, and in particular, producing compositions comprising polyunsaturated fatty acids wherein the degradation of the polyunsaturated fatty acids is inhibited, suppressed or reduced.

### BACKGROUND OF THE INVENTION

Polyunsaturated fatty acids (including long chain polyunsaturated fatty acids (LC-PUFAs)), and especially long chain omega-3 fatty acids (e.g., docosahexanoic acid (DHA) and eicosapentaenoic acid (EPA)), are known to enhance cognitive function and maintain cardiovascular health, among other health benefits (See, e.g., von Schacky, C., “Omega-3 Fatty Acids and Cardiovascular Disease,” Current Opinion in Clinical Nutrition and Metabolic Care 7, no. 2 (March 2004): 131-6 and Simopoulos, A. P., “Essential Fatty Acids in Health and Chronic Disease,” American Journal of Clinical Nutrition 79, no. 3 (March 2004): 523-4.) Recent qualified studies have also indicated that omega-3 fatty acids are effective in reducing the risk of coronary heart disease (See “FDA Announces Qualified Health Claims for Omega-3 Fatty Acids,” FDA News, Sep. 8, 2004, www.fda.gov/bbs/topics/news/2004/NEW01115.html). In addition, consumer trends indicate demands for products containing polyunsaturated fatty acids are increasing.

Essential fatty acids such as omega-3 fatty acids are nutrients required in the human diet. However, omega-3 fatty acids are not synthesized in human body, but are found in natural sources such as the oil of certain plants and animals, including fishes, walnuts, lingonberrys, hemp, flax, chia, perilla, purslane, and algae. Since omega-3 fatty acids are not synthesized by the body, they, and their health benefits, must be obtained through food or dietary supplement. Supplementing a diet with omega-3 fatty acids frequently involves ingestion of supplements which have a fishy odor and/or taste.

Within the body, omega-3 fatty acids are modified to make eicosanoids, which affect inflammation and other cellular functions, endogenous cannabinoids, which affect mood, behavior, and inflammation, resolvins, isofurans, isoprostanes, epoxyeicosatrienoic acids (EETs), and neuroprotectin D. In addition, omega-3 fatty acids form lipid rafts affecting cellular signaling and act on DNA to activate or inhibit transcription factors for NFκB, a pro-inflammatory cytokine.

During storage under certain conditions, polyunsaturated fatty acids can become unstable and degrade. Thus, various means of incorporating polyunsaturated fatty acids into functional food and beverage products have been used to try to reduce or eliminate degradation of polyunsaturated fatty acids for delivery to a consumer. For example, products have been produced as bulk oils (for spread and softgel capsules), powdered omega-3 (for cereal bars), microencapsulated omega-3 oils (for cereal bars, yogurt and beverages) and liposome/emulsion concentrates (for beverages). Technology for dispersion of omega-3 fatty acids in food using whey protein as an emulsifier and technologies using high oil loading liposome to deliver polyunsaturated fatty acids have also been developed.

However, convention emulsion technology, i.e., homogenizing the omega-3 fatty oils using food grade emulsifier (e.g., gum arabic or lecithin), gives unstable emulsions with large oil particle sizes. Maintaining both physical and chemical stability of polyunsaturated fatty acids in compositions such as beverages is particularly difficult because polyunsaturated fatty acids are prone to oxidation, which can adversely impact the organoleptic properties of these compositions. In addition, such oxidation is undesirable according to recent research because consumption of foods containing highly oxidized lipids may have adverse health implications.

Furthermore, processing of typical emulsions by homogenization of the compositions comprising the polyunsaturated fatty acids requires large mixing equipment, storage, and transport requirements to facilitate delivery of these fatty acid emulsions to the consumer. Therefore, it would be desirable to provide omega-3 fatty acids to consumers without the acid becoming unstable or degrading.

### SUMMARY OF THE INVENTION

The embodiments of the present invention provides an emulsion comprising a continuous liquid phase, an emulsifier, and a discontinuous liquid phase comprising a blend including a polyunsaturated fatty acid source and a dispersing agent. The polyunsaturated fatty acid source comprises at least one polyunsaturated fatty acid and the weight ratio of the fatty acid source to the dispersing agent in the blend ranges from about between 9:1 and 1:10.

In addition, embodiments of the present invention provides a method for making an emulsion comprising providing a first liquid, providing a second liquid comprising a blend including a long chain polyunsaturated fatty acid source and a dispersing agent, and combining the first liquid, the second liquid, and an emulsifier so as to form the emulsion comprising a continuous liquid phase including the first liquid and a discontinuous liquid phase including the second liquid. The weight ratio of the polyunsaturated fatty acid source to the dispersing agent in the blend ranges from about between 9:1 and 1:10 and the long chain polyunsaturated fatty acid source comprising at least one long chain polyunsaturated fatty acid.

Furthermore, embodiments of the present invention include a method for inhibiting, suppressing, or reducing at least a portion of any degradation of at least one omega-3 fatty acid in a composition, the method comprising providing at least one polyphenol in the composition.

Additionally, embodiments of the present invention include an emulsion comprising a continuous liquid phase, an emulsifier, and a discontinuous liquid phase. The discontinuous liquid phase comprises a blend including a polyunsaturated fatty acid source, a weighing agent, and a dispersing agent. The polyunsaturated fatty acid source comprises at least one polyunsaturated fatty acid.

Other objects, features, and advantages of this invention will be apparent from the following detailed description, drawing, and claims.

### BRIEF DESCRIPTION OF THE DRAWINGS

FIG. 1 is a graph of the particle diameter of the emulsions formed in EXAMPLE 2.

### DETAILED DESCRIPTION

As summarized above, this disclosure encompasses emulsions comprising polyunsaturated fatty acids and a method for making such an emulsion. This disclosure also encompasses emulsions such as, but not limited to, beverages, herbal compositions, or pharmaceutical compositions, and combinations thereof. The formation of a stable emulsion according to embodiments of the present invention inhibits, reduces, or suppresses the oxidation, and the associated fishy odor and smell, of the LC-PUFAs. In particular embodiments, the emulsion comprises an emulsion concentrate. By producing embodiments of the present invention as beverage emulsion concentrates, beverages and the like, polyunsaturated fatty acids, and particularly LC-PUFAs, and their health benefits, may be provided to the consumer in a stable and well dispersed form.

As used herein, “emulsion concentrate” refers to an emulsion which may be used to produce a final product emulsion having lower concentrations of emulsifier and discontinuous liquid phase than the concentrations of emulsifier and discontinuous liquid phase in the emulsion concentrate. For example, the emulsion concentrate may comprise a beverage emulsion concentrate which can be used to form a beverage. In particular, the emulsion concentrate is easily dispersed within a continuous liquid phase, without further homogenization required. The formation of emulsion concentrates allows for the storage of LC-PUFAs in a stable and compact form for storage as well as transport before being dosed into a final emulsion form for consumption by a consumer. In addition, homogenization of the emulsion concentrate may be carried out in a smaller scale than homogenization of a final emulsion form to be consumed by a consumer. Thus, lower equipment costs are realized.

Embodiments of the emulsion of the present invention comprise a continuous liquid phase, an emulsifier, and a discontinuous liquid phase. As used herein, “emulsion” refers to an immiscible mixture of a continuous liquid phase and a discontinuous liquid phase. As used herein, “continuous liquid phase” refers to the portion of the emulsion in which the discontinuous liquid phase is dispersed. Accordingly, “discontinuous liquid phase,” as used herein, refers to the multiplicity of discrete elements dispersed within, and immiscible with, the continuous liquid phase. In addition, embodiments of the discontinuous liquid phase include a blend including a dispersing agent and a LC-PUFA source. The LC-PUFA source includes at least one LC-PUFA. As used herein, “dispersing agent” refers to any material which increases the stability of emulsions of the present invention and/or increases the ease of dispersion of discontinuous liquid phases of the present invention within continuous liquid phases. As used herein, “LC-PUFA” refers to any polyunsaturated carboxylic acid or organic acid with a long aliphatic tail. It should also be understood by a person of ordinary skill in the art that though the embodiments described herein which include LC-PUFA may include other polyunsaturated fatty acids such as short chain polyunsaturated fatty acids or medium chain polyunsaturated fatty acids instead of or in combination with the LC-PUFA.

As used herein, “emulsifier” refers to any substance which increases the stability of the emulsion so that the discontinuous liquid phase remains substantially dispersed within the continuous liquid phase once the emulsion is formed. Within particular embodiments of the emulsion of the present invention, the emulsifier may be at least partially soluble in the continuous liquid phase, the discontinuous liquid phase, or both.

In embodiments of the present invention, the continuous liquid phase may be any liquid which is compatible with the LC-PUFA, the discontinuous liquid phase, and the emulsifier. In some embodiments, the continuous liquid phase may be, but is not limited to, a consumer product capable of ingestion so as to provide for delivery of the LC-PUFA to a consumer. Thus, according to particular embodiments of the present invention, the continuous liquid phase may include, but is not limited to, water, carbonated water, syrup, diet beverages, carbonated soft drinks, fruit juices, vegetable juices, isotonic beverages, non-isotonic beverages, soft drinks containing fruit juice, coffee, tea, other aqueous liquids, pharmaceutical excipients, natural sweeteners, synthetic sweeteners, caloric sweeteners, non-caloric sweeteners, sodium benzoate, ethylenediaminetetraacetic acid (EDTA), ascorbic acid, citric acid, dietary fiber, dairy products, soy products, and the like, and combinations thereof

In particular embodiments, the continuous liquid phase may be acidic. In one embodiment, the continuous liquid phase may have a pH between 2 and 7 In another embodiment, the continuous liquid phase may have a pH between 2.5 and 5.

In particular embodiments, the continuous liquid phase includes at least one polyphenol. The polyphenol may inhibit, suppress, or reduce degradation of the LC-PUFA and prevents lipid oxidation. Thus, the polyphenol may also prevent any odor or taste of the LC-PUFA from being perceived by a consumer. In addition, polyphenols have also been noted as being effective in protecting against cardiovascular diseases and cancer (See Arts and Hollman, “Polyphenols and Disease Risk in Epidemiologic Studies,” Am J Clin Nutr 2005;81 (suppl): 317S-25S).

Examples of suitable polyphenols for embodiments of this invention include, but are not limited to, polyphenols found naturally in a variety of foods including plants, tea leaves, fruits, vegetables, and cocoa or may be synthesized or synthetic. For example, the polyphenol may comprise a phenolic acid or a flavonoid. Examples of phenolic acids include, but are not limited to, cinnamic acid or benzoic acid. Flavonoids which may be used with embodiments of this invention included flavonols, flavones, flavanones, flavanols, isoflavones, anthocyanidins, tannins, and stilbenes, for example.

In particular embodiments, the polyphenol may comprise a flavonoid such as quercetin, proanthocyanidin, catechin, resveratrol, and procyanidin, for instance. In other embodiments, the polyphenol may comprises a catechin selected from the group consisting of (+)-catechin, (−)-epicatechin, (−)-epicatchin gallate, (−)-epigallocatechin, and epigallocatechin gallate. In particular embodiments, suitable polyphenols may be included in the emulsions in commercial available antioxidants such as the antioxidants listed in Table 1 below:

TABLE 1

| | | Active | Purity |
|---|---|---|---|
| Antioxidant/Source | Supplier | Ingredient | (%) |
| Vitamin C | N/A | Ascorbic Acid | 99 |
| AQ-3000 | San-Ei Gen F.F.I | Enzyme Modified | 10 |
| (EMIQ) | | Isoquercitrin (EMIQ) | |
| Alpha-Lipoic acid/ | AquaNova | Alpha-Lipoic acid | 10 |
| Nanoemulsion | | | |
| Co-enzyme Q10/ | AquaNova | Co-enzyme Q10 | 22 |
| Nanoemulsion | | | |
| Sunphenon, ECGC | Taiyo | Epigallocatechin-3- | 90 |
| Taiyo Green Tea | | gallate (ECGC) | |
| Polyphenols | | | |
| Sunphenon, 90M | Taiyo | Total Polyphenols | 80 |
| Taiyo Green Tea | | | |
| Polyphenols | | | |
| Chinese Green | China ChengDu IM/EX | Total Polyphenols | 40 |
| Tea Polyphenols, T40 | | | |
| Chinese Green | China ChengDu IM/EX | Total Polyphenols | 80 |
| Tea Polyphenols, T80 | | | |
| Grape Seed Extract, H, | Cargill | Proanthocyanidin | 65 |
| #1 | | (Total Polyphenols) | (95) |
| Grape Seed Extract, # 2 | PL Thomas | Proanthocyanidin | 60 |
| | | (Total Polyphenols) | (90) |
| Quercetin Dehydrated | PL Thomas | Quercetin | 96 |
| Citrus Bioflavonoid | PL Thomas | Flavonoids | 46 |
| Complex | | | |
| Pomegranate | PL Thomas | Punicosides | 40 |
| | | (Total Polyphenols) | (80) |
| Appol | PL Thomas | Total Polyphenols | 55 |
| Apple Extract | | | |
| White Cherry PE | PL Thomas | Total Polyphenols | 50 |
| Elderberry | PL Thomas | Total Polyphenols | 30 |
| Prune PE, Plum Extract | PL Thomas | Total Polyphenols | 50 |
| VivOX, | PL Thomas | Carnosic Acid | 45 |
| Rosemary Extract | | | |
| Curcumin | PL Thomas | Curcumin | 96 |
| Pyncogenol | Dr. Cranton | Proanthocyanidins | N/A |
| Pine-bark extract | | | |
| Origanox WS | Barrington Chemical | Rosmarinic Acid | 7 |
| | | Phenolic compounds | 16 |
| Wolfberry | Da Li | N/A | N/A |
| Taurine | Sigma | Taurine | 98 |
| Caffeine | N/A | Caffeine | 99 |

According to particular embodiments of the invention, the polyphenol may be present in the emulsion in an amount ranging from about 0.01% by weight of the emulsion to about 10% by weight of the emulsion. More particularly, the polyphenol may be present in the emulsion in an amount ranging from about 0.01% by weight of the emulsion to about 5% by weight of the emulsion. Still more particularly, the polyphenol may be present in the emulsion in an amount ranging from about 0.1% by weight of the emulsion to about 3% by weight of the emulsion.

In particular embodiments, the continuous liquid phase may additionally include a water dispersible bioactive. As used herein, “water dispersible bioactive” refers to materials which are both dispersible in water and soluble in water. Suitable water dispersible bioactives for embodiments of the present invention include, but are not limited to, lutein, β-carotene, lycopene (e.g., from tomato), astaxanthin, zeaxanthin, enzymes such as papain (e.g., from papaya), carotenoids (e.g., from watercress), eucalyptol (e.g., from basil or rosemary), eugerol (e.g., from basil), gingerol (e.g., from ginger), avenacoside (e.g., from oats), phenolic acids such as gallic acid (e.g., from blueberries) or rosmarinic acid (e.g., from rosemary), flavonoids (e.g., from watercress or willow) such as quercetin (e.g., from blueberries, grape seeds, grapes, mate, or green tea), catechins (e.g., from green tea), anthocyanins (e.g., from grape seeds, grapes, or blueberries), phytoestrogen (e.g., from red clover), or naringin (e.g., from grapefruit), coumarins (e.g., from oats), proanthocyanidins (e.g., from grape seeds, green tea, guarana, or mate), curcuminoids (e.g., from tumeric), caramel coloring, vitamins such as Vitamin E (e.g., from cucumber) or Vitamin K (e.g., from alfalfa), and combinations thereof or any natural or synthetic food grade colored or uncolored material which absorbs UV light or any other material understood by a person of ordinary skill in the art to be a suitable water dispersible bioactive, for example. Additional water dispersible bioactives which may be used in embodiments of the present invention are found in “Lipid Oxidation”, by E. N. Frankel, pages 209-298, 2^{nd} Edition, The Oily Press, 2005, which is hereby incorporated by reference. According to particular embodiments of the invention, the water dispersible bioactives may be present in the continuous liquid phase in an amount ranging from about 0% by weight of the continuous liquid phase to about 20% by weight of the continuous liquid phase. According to other embodiments of the invention, the water dispersible bioactives may be present in the continuous liquid phase in an amount ranging from about 50 mg to about 100 mg.

In some embodiments and without being bound by theory, the water dispersible bioactives provide photo-oxidative protection such that the oxidation of the polyunsaturated fatty as is reduced, inhibited or suppressed. It is believed that the water soluble bioactive absorbs some UV light such that polyunsaturated fatty acid is exposed to less light. In some embodiments of the present in invention which are ingestible, the water soluble bioactives may hydrate a portion of the skin of the consumer ingesting the emulsion.

Embodiments of the present invention also include a discontinuous liquid phase which is capable of being dispersed within the continuous liquid phase and which comprises a blend including a LC-PUFA source and a dispersing agent. The discontinuous liquid phase is immiscible in the continuous liquid phase.

Suitable LC-PUFA sources for embodiments of the present invention include any LC-PUFA source which comprises at least one LC-PUFA capable of being dispersed in an emulsion. According to particular embodiments of the invention, the LC-PUFA source may be a LC-PUFA oil or a LC-PUFA powder, or combinations thereof. Suitable LC-PUFA oils can be derived from algae, fish, animals, plants, or combinations thereof, for example. In such embodiments of the emulsion comprising a LC-PUFA oil, the blend may be referred to herein as an “oil blend”. LC-PUFA oils for embodiments of the present invention include omega-3 fatty acid oils, omega-6 fatty acid oils and omega-9 fatty acid oils, for instance. Examples of suitable omega-3 fatty acid oils for embodiments of this invention include, but are not limited to, alpha-linolenic acid oil, eicosapentaenoic acid oil, docosahexaenoic acid oil, and combinations thereof. In particular embodiments, the omega-3 fatty acid may be synthesized. Suitable omega-6 fatty acid oils for embodiments of this invention include, but are not limited to, gamma-linolenic acid oil, and arachidonic acid oil. In some embodiments, suitable omega-3 fatty acid oils include fish oils, (e.g., menhaden oil, tuna oil, salmon oil, bonito oil, and cod oil), microalgae docosahexaenoic acid oil, microalgae omega-S oils, and the like, or combinations thereof. The fish oils may be crude or refined and also may be enzyme treated. In particular embodiments, suitable omega-3 fatty acid oils may include commercially available omega-3 fatty acid oils such as Microalgae DHA oil (from Martek, Columbia, Md.), OmegaPure (from Omega Protein, Houston, Tex.), Marinol C-38 (from Lipid Nutrition, Channahon, Ill.), Bonito oil and MEG-3 (from Ocean Nutrition, Dartmouth, NS), Evogel (from Symrise, Holzminden, Germany), Marine Oil, from tuna or salmon (from Arista Wilton, Conn.), OmegaSource 2000, Marine Oil, from menhaden and Marine Oil, from cod (from OmegaSource, RTP, N.C.).

In particular embodiments wherein the emulsion comprises an emulsion concentrate, the LC-PUFA source is present in the emulsion concentrate in an amount ranging from about 0.5% by weight of the emulsion concentrate to about 35% by weight of the emulsion concentrate. More particularly, the LC-PUFA source is present in the emulsion concentrate in an amount ranging from about 2% by weight of the emulsion concentrate to about 30% by weight of the emulsion concentrate. Still more particularly, the LC-PUFA source is present in the emulsion concentrate in an amount ranging from about 5% by weight of the emulsion concentrate to about 20% by weight of the emulsion concentrate. Still more particularly, the LC-PUFA source is present in the emulsion concentrate in an amount ranging from about 15% by weight of the emulsion to about 20% by weight of the emulsion concentrate.

In particular embodiments, the LC-PUFA source is present in the emulsion in an amount ranging from about 0.002% by weight of the emulsion to about 35% by weight of the emulsion. More particularly, the LC-PUFA source is present in the emulsion in an amount ranging from about 0.005% by weight of the emulsion to about 30% by weight of the emulsion. Still more particularly, the LC-PUFA source is present in the emulsion in an amount ranging from about 0.01% by weight of the emulsion to about 20% by weight of the emulsion.

In some embodiments, the dispersing agent is selected from vitamin E, ascorbyl palmitate, rosemary extract, a terpene, a flavor oil, a vegetable oil, or an essential oil and the like, and combinations thereof. According to particular embodiments the essential oil may be a citrus oil, leaf oil, spice oil, peel oil, and combinations thereof. Examples of suitable essential oils for embodiments of this invention include, but are not limited to, lemon oil, orange oil, lime oil, grapefruit oil, mandarin oil, bitter orange oil, mint oil, peppermint oil, rosemary oil, flax seed oil, cranberry seed oil, bergamot oil, and combinations thereof. In embodiments where the dispersing agent comprises a terpene, suitable terpenes include, but are not limited to, d-limonene, 1-limonene, dl-limonene (i.e., greater than 99 wt % dl-limonene), orange distillate oil (i.e., greater than 97 wt % dI-limonene) and combinations thereof.

In some embodiments, the blend may additionally include a weighing agent. Suitable weighing agents for embodiments of the present invention include brominated vegetable oil, ester gum and other wood rosins, sucrose diacetate hexa-isoburtyurate (SAIB), refined gum dammar, ganuaba wax, benzyl benzoate. polyglyceryl ester, glyceryl tribenzoate, and combinations thereof, for example. In particular embodiments, the continuous liquid phase further comprises a sugar. Examples of suitable sugars for embodiments of the present invention include a monosaccharide, a disaccharide, a trisaccharide, an oligosaccharide, or combinations thereof. Examples of continuous liquid phases which include a sugar include carbonated beverages with caloric sweeteners, fruit juices, and combinations thereof.

In some embodiments, the continuous liquid phase may also include a high-potency sweetener. Examples of suitable high-potency sweeteners include dulcoside A, dulcoside B, rubusoside, stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, cyclocarioside I, sucralose, acesulfame potassium or other salts, aspartame, alitame, saccharin, neohesperidin dihydrochalcone, cyclamate, neotame, N-[N-[3-(3-hydroxy-4-methoxyphenyl)propyl]-L-α-aspartyl]-L-phenylalanine 1-methyl ester, N-[N-[3-(3-hydroxy-4-methoxyphenyl)-3-methylbutyl]-L-α-aspartyl]-L-phenylalanine 1-methyl ester, N-[N-[3 -(3 -methoxy-4-hydroxyphenyl)propyl]-L-α-aspartyl]-L-phenylalanine 1-methyl ester, salts thereof, and combinations thereof.

Without being bound by theory, the weighing agent in such embodiments increases the density of the discontinuous liquid phase so that the discontinuous liquid phase does not float to the top of the emulsion and agglomerate. Such functionality is particularly useful in embodiments where the continuous liquid phase contains sugar, which may increase the density of the continuous liquid phase. Thus, when the density of the continuous liquid phase is increased by the presence of sugar, the disparity of the densities of the continuous and discontinuous liquid phases is increased, resulting in the less dense discontinuous liquid phase having a tendency to rise to the top of the emulsion if the weighing agent is not present.

In particular embodiments, the weighing agent is present in the discontinuous liquid phase in an amount ranging from about 1% to about 50% of the discontinuous liquid phase. In other embodiments, the weighing agent is present in the discontinuous liquid phase in an amount ranging from about 5% to about 35% of the discontinuous liquid phase.

In embodiments where the weighing agent comprises brominated vegetable oil (BVO), the weighing agent is present in the discontinuous liquid phase in an amount ranging from about 1% to about 30% of the discontinuous liquid phase. More specifically, the brominated vegetable oil (BVO) weighing agent may be present in the discontinuous liquid phase in an amount ranging from about 5% to about 20% of the discontinuous liquid phase. In embodiments where the weighing agent comprises glycervl ester of wood rosin (i.e., ester gum), the weighing agent is present in the discontinuous liquid phase in an amount ranging from about 1% to about 50% of the discontinuous liquid phase. More specifically, the glyceryl ester of wood rosin weighing agent may be present in the discontinuous liquid phase in an amount ranging from about 5% to about 35% of the discontinuous liquid phase. In embodiments where the weighing agent comprises sucrose diacetate hexa-isobutyrate (SAIB), the weighing agent is present in the discontinuous liquid phase in an amount ranging from about 1% to about 50% of the discontinuous liquid phase. More specifically, the sucrose diacetate hexa-isobutyrate weighting agent may be present in the discontinuous liquid phase in an amount ranging from about 5% to about 35% of the discontinuous liquid phase. In embodiments where the weighing agent comprises refined gum damar, the weighing agent is present in the discontinuous liquid phase in an amount ranging from about 1% to about 50% of the discontinuous liquid phase. More speciflically, the refined gum damar weighing agent may be present in the discontinuous liquid phase in an amount ranging from about 5% to about 35% of the discontinuous liquid phase. In embodiments where the weighing agent comprises ganuaba wax, the weighing agent is present in the discontinuous liquid phase in an amount ranging from about 1% to about 50% of the discontinuous liquid phase. More specifically, the ganuaba wax weighing agent may be present in the discontinuous liquid phase in an amount ranging from about 5% to about 35% of the discontinuous liquid phase. In embodiments where the weighing agent comprises benzyl benzoate, the weighing agent is present in the discontinuous liquid phase in an amount ranging from about 1% to about 40% of the discontinuous liquid phase. More specifically, the benzyl benzoate weighing agent may be present in the discontinuous liquid phase in an amount ranging from about 5% to about 30% of the discontinuous liquid phase. In embodiments where the weighing agent comprises polyglyceryl ester, the weighing agent is present in the discontinuous liquid phase in an amount ranging from about 1% to about 50% of the discontinuous liquid phase. More specifically.. the polyglyceryl ester weighing agent may be present in the discontinuous liquid phase in an amount ranging from about 5% to about 35% of the discontinuous liquid phase. In embodiments where the weighing agent comprises glyceryl tribenzoate, the weighing agent is present in the discontinuous liquid phase in an amount ranging from about 1% to about 30% of the discontinuous liquid phase. More specifically, the glyceryl tribenzoate weighing agent may be present in the discontinuous liquid phase in an amount ranging from about 5% to about 25% of the discontinuous liquid phase.

Generally, the amount of dispersing agent in the blend should be sufficient to create a stable emulsion rather than just masking the flavor or smell of the LC-PUFA. In particular, the amount of dispersing agent should be sufficient to provide oxidative stability (i.e., inhibit, suppress, or reduce oxidation of the LC-PUFA) and stabilize the resulting emulsion. In some embodiments, the weight ratio of the LC-PUFA source to the dispersing agent in the blend can range from about 9:1 to about 1:10. More particularly, the weight ratio of the LC-PUFA source to the dispersing agent in the blend can range from about 5:1 to about 1:1. Still more particularly, the weight ratio of the LC-PUFA source to the dispersing agent in the blend can range from about 4:1 to about 3:1.

In particular embodiments wherein the emulsion comprises an emulsion concentrate, the blend may be present in the emulsion concentrate in an amount ranging from about 0.5% by weight of the emulsion concentrate to about 35% by weight of the emulsion concentrate. More particularly, the blend is present in the emulsion in an amount ranging from about 2% by weight of the emulsion concentrate to about 30% by weight of the emulsion concentrate. Still more particularly, the blend is present in the emulsion concentrate in an amount ranging from about 5% by weight of the emulsion concentrate to about 20% by weight of the emulsion concentrate; Still more particularly, the blend is present in the emulsion concentrate in an amount ranging from about 10% by weight of the emulsion concentrate to about 20% by weight of the emulsion concentrate.

In particular embodiments, the blend may present in the emulsion in an amount ranging from about 0.001% by weight of the emulsion to about 35% by weight of the emulsion. More particularly, the blend may be present in the emulsion in an amount ranging from about 0.005% by weight of the emulsion to about 30% by weight of the emulsion. Still more particularly, the blend may be present in the emulsion in an amount ranging from about 0.01% by weight of the emulsion to about 20% by weight of the emulsion. Still more particularly, the blend may be present in the emulsion in an amount ranging from about 0.02% by weight of the emulsion to about 20% by weight of the emulsion.

In some embodiments, the blend may further comprise a folded oil. In particular embodiments, the folded oils further improve the oxidative stability and reduces improves the particle size distribution by reducing the particle size of the discontinuous liquid phase. Suitable folded oils for embodiments of the present invention include, but are not limited to, 4-fold bergamot oil, bergaptene free bergamot oil, terpeneless grapefruit oil, 4-fold grapefruit oil, 5-fold grapefruit oil, 6-fold grapefruit oil, 10-fold grapefruit oil, high aldehyde grapefruit oil, 5-fold grapefruit juice extract, 7-fold grapefruit juice extract, terpeneless lemon oil, 2-fold lemon oil, 3-fold lemon oil, 5-fold lemon oil, 10-fold lemon oil, 13-fold lemon oil, washed 5-fold lemon oil, 10-fold lemon oil, Sesquiterpeneless lemon oil, FC free lemon oil, distilled 3-fold lime oil, distilled 4-fold lime oil, distilled 5-fold lime oil, distilled terpeneless lime oil, distilled sesquiterpeneless lime oil, distilled washed 5 fold lime oil, cold pressed 3-fold lime oil, cold pressed 4-fold lime oil, cold pressed 5-fold lime oil, cold pressed 10-fold lime oil, cold pressed terpeneless lime oil, 4-fold mandarin oil, 5-fold mandarin oil. 10-fold mandarin oil, terpeneless orange oil, 2-fold orange oil, 3-fold orange oil, 4-fold orange oil, 5-fold orange oil, 7-fold orange oil, 8-fold orange oil, 10-fold orange oil, 15-fold orange oil, 20-fold orange oil, 25-fold orange oil, 30-fold orange oil, 5-fold orange juice extract, 8-fold orange juice extract, 3-fold tangerine oil, 5-fold tangerine oil, terpeneless tangerine oil, and combinations thereof. Thus, in some embodiments, the discontinuous liquid phase may comprise a terpeneless oil.

According to particular embodiments of the invention, the folded oil may be present in the discontinuous liquid phase in an amount ranging from about 1% by weight of the discontinuous liquid phase to about 60% by weight of the discontinuous liquid phase. More particularly, the folded oil may be present in the discontinuous liquid phase in an amount ranging from about 7.5% by weight of the discontinuous liquid phase to about 45% by weight of the discontinuous liquid phase. Still more particularly, the folded oil may be present in the discontinuous liquid phase in an amount ranging from about 10% by weight of the discontinuous liquid phase to about 40% by weight of the discontinuous liquid phase.

In alternate embodiments of the present invention, the emulsion may comprise a continuous liquid phase, an emulsifier, and a discontinuous liquid phase comprising a blend including a polyunsaturated fatty acid source and a folded oil. Thus, in particular alternate embodiments of the present invention, the emulsion might not include a dispersing agent. Rather, the folded oil helps to form a stable emulsion wherein the degradation of the polyunsaturated fatty acid is inhibited, suppressed, or reduced.

In particular embodiment, the discontinuous liquid phase may also include medium chain triglycerides. In particular embodiments, the medium chain triglycerides further improve the oxidative stability and reduces improves the particle size distribution by reducing the particle size of the discontinuous liquid phase. According to particular embodiments of the invention, the medium chain triglycerides may be present in the discontinuous liquid phase in an amount ranging from about 1% by weight of the discontinuous liquid phase to about 60% by weight of the discontinuous liquid phase. More particularly, the medium chain triglycerides may be present in the discontinuous liquid phase in an amount ranging from about 7.5% by weight of the discontinuous liquid phase to about 40% by weight of the discontinuous liquid phase. Still more particularly, the medium chain triglycerides may be present in the discontinuous liquid phase in an amount ranging from about 10% by weight of the discontinuous liquid phase to about 30% by weight of the discontinuous liquid phase.

In some embodiments, the discontinuous liquid phases may also include other components such as oil soluble vitamins (e.g., vitamin A, vitamin D, vitamin E, or Vitamin K), phytochemicals, and other lipid nutrients.

In particular embodiments, the discontinuous liquid phase may additionally include an oil dispersible bioactive. As used herein, “soil dispersible bioactive” refers to materials which are both dispersible in oil and soluble in oil. Suitable oil dispersible bioactives for embodiments of the present invention include, but are not limited to, oxygenated carotenoids, such as lutein (e.g., from tomato), astaxanthin and non-oxygenated carotenoids, such as β-carotene and lycopene, and combinations thereof or any natural or synthetic food grade colored or uncolored material which absorbs UV light, for example. In other embodiments, suitable oil dispersible bioactives may include enzymes such as papain (e.g., from papaya), carotenoids (e.g., from watercress), eucalyptol (e.g., from basil or rosemary), eugerol (e.g., from basil), gingerol (e.g., from ginger), avenacoside (e.g., from oats), phenolic acids such as gallic acid (e.g., from blueberries) or rosmarinic acid (e.g., from rosemary), flavonoids (e.g., from watercress or willow) such as quercetin (e.g., from blueberries, grape seeds, grapes, mate, or green tea), catechins (e.g., from green tea), anthocyanins (e.g., from grape seeds, grapes, or blueberries), phytoestrogen (e.g., from red clover), or naringin (e.g., from grapefruit), coumarins (e.g., from oats), proanthocyanidins (e.g., from grape seeds, green tea, guarana, or mate), curcuminoids (e.g., from tumeric), caramel coloring, and any other material understood by a person of ordinary skill in the art to be a suitable water dispersible bioactive, for instance. Additional oil dispersible bioactives which may be used in embodiments of the present invention are found in “Lipid Oxidation”, by E. N. Frankel, pages 209-298, 2^{nd} Edition, The Oily Press, 2005. According to particular embodiments of the invention, the oil dispersible bioactives may be present in the discontinuous liquid phase in an amount ranging from about 0% by weight of the discontinuous liquid phase to about 20% by weight of the discontinuous liquid phase. According to other embodiments of the invention, the oil dispersible bioactives may be present in the discontinuous liquid phase in an amount ranging from about 50 mg to about 100 mg. In some embodiments and without being bound by theory, the oil dispersible bioactives provide photo-oxidative protection such that the oxidation of the polyunsaturated fatty as is reduced, inhibited or suppressed. It is believed that the oil dispersible bioactive absorbs some UV light such that polyunsaturated fatty acid is exposed to less light. In some embodiments of the present in invention which are ingestible, the oil soluble bioactives may hydrate a portion of the skin of the consumer ingesting the emulsion.

In other embodiments, the discontinuous liquid phase may also include an oil blend antioxidant. Suitable oil blend antioxidants for embodiments of the present invention include, but are not limited to, butylated hydroxytoluene (BHT), butylated hydroxyanisole (BHA), tert-butylhydroquinone (TBHQ), and combinations thereof. According to particular embodiments of the invention, the oil blend antioxidant may be present in the discontinuous liquid phase in an amount ranging from about 0% by weight of the discontinuous liquid phase to about 5% by weight of the discontinuous liquid phase.

Embodiments of the present invention include emulsions in which the discontinuous liquid phase is present in the emulsion in the form of particles. In some embodiments, these discontinuous liquid phase particles have an average particle size between 0.1 μm and 1.5 μm. More particularly, the discontinuous liquid phase particles may have an average particle size between 0.1 μm and 1.0 μm. Still more particularly, the discontinuous liquid phase particles may have an average particle size between 0.15 μm and 0.7 μm.

Emulsifiers which may be used in the present invention include any emulsifier compatible with the LC-PUFAs and the dispersing agents used in the emulsion. Natural or synthetic emulsifiers may be suitable for embodiments of the present invention. According to particular embodiments of the present invention, the emulsifier may be a modified natural emulsifier. That is, the emulsifier may be chemical modified, enzymatically modified, physically modified, or combinations thereof. In embodiments where the emulsion is used in a consumer composition such as a beverage, the emulsifier is a food grade emulsifier. Examples of other suitable emulsifiers for embodiments of this invention include, but are not limited to, gum arabic, pectin, p-pectin, gum ghatti, modified gum arabic (e.g., Ticamulsion™, from TIC Gums, Beleamp, Md.), gum acacia (e.g., Eficacia™, from Colloidies Naturels International (CNI), Bridgewater, N.J.), modified food starch (e.g., from National Starch & Chemical, Bridgewater, N.J.), polysorbates (i.e., tweens), co-emulsifiers such as propylene glycol alginate (PGA), and combinations thereof.

In embodiments where the emulsion comprises a fruit juice or drink containing a fruit juice (e.g., orange juice or grapefruit juice), citrus oil may be present in the continuous liquid phase, which aids in the chemical stability of the emulsion and thus, a dispersing agent need not be additionally added to the emulsion. In particular embodiments wherein the continuous liquid phase comprises an acidic composition, such as a carbonated beverage, the emulsifier may comprise a carbohydrate-based macromolecule. Examples of suitable carbohydrate-based macromolecules include gum acacia, modified food starch, gum ghatti, pectins (e.g., beta-pectin), modified gum acacia, and combinations thereof.

In particular embodiments, the emulsifier is present in the emulsion in an amount ranging from about 0.0002% by weight of the emulsion to about 45% by weight of the emulsion. In other embodiments, the emulsifier is present in the emulsion in an amount ranging from about 0.001% by weight of the emulsion to about 25% by weight of the emulsion. In still other embodiments, the emulsifier is present in the emulsion in an amount ranging from about 0.01% by weight of the emulsion to about 20% by weight of the emulsion. In still other embodiments, the emulsifier is present in the emulsion in an amount ranging from about 5% by weight of the emulsion to about 20% by weight of the emulsion.

In some embodiments, the emulsion may also include a stabilizing agent to further stabilize the emulsion and also improve the taste profile and/or improve the shelf life of the emulsion. Examples of suitable stabilizing agents for embodiments of this invention include, but are not limited to, vitamin C, polyphenols from fruit and vegetable sources, such as rosemary extract, tea polyphenols and grape seed extracts, ethylenediaminetetraacetic acid (EDTA), ethylenediaminetetraacetic acid disodium salt, and combinations thereof. Within particular embodiments of the present invention, the stabilizing agent may be at least partially soluble in the continuous liquid phase, the discontinuous liquid phase, or both. In one embodiment, an emulsion comprising a stabilizing agent has a shelf life of more than 3 months. As used herein, “shelf-life” refers to a time period within which embodiments of emulsions may be stored and remain suitable for consumer use.

In other embodiments, the emulsion further comprises a surfactant which further reduces the surface tension between the oil phase and the suspension medium, thereby improving the stability of the emulsion and homogenization of the emulsion. Examples of suitable surfactants for embodiments of this invention include, but are not limited to, dioctyl succinate sulfate sodium salt (DSS), medium chain triglyceride (MCT), propylene glycol alginate (PGA) and combinations thereof Within particular embodiments of the present invention, the surfactant may be at least partially soluble in the continuous liquid phase, the discontinuous liquid phase, or both.

According to particular embodiments of the invention, methods for making an emulsion comprise providing a first liquid, providing a second liquid, and combining the first liquid, the second liquid, and an emulsifier so as to form the emulsion. Embodiments of the second liquid comprise a blend that includes a long chain polyunsaturated fatty acid source and a dispersing agent. Particular embodiments of long chain polyunsaturated fatty acid sources include at least one long chain polyunsaturated fatty acid. In particular embodiments, emulsions comprise a continuous liquid phase including the first liquid and a discontinuous liquid phase including the second liquid. Embodiments of the emulsion may be any of the emulsions described above.

In particular embodiments of the present invention, the first liquid may comprise the same components suitable for embodiments of the continuous liquid phases described above. Embodiments of the second liquid may include dispersing agents similar to any of the dispersing agents above. In addition, LC-PUFA sources included in embodiments of the second liquid may be any of the LC-PUFA sources described above. Furthermore, embodiments of the second liquid may comprise the same components suitable for embodiments of the discontinuous liquid phases described above. Suitable emulsifiers may be similar to the emulsifiers described above.

Embodiments of the present invention include methods wherein the step of combining comprises combining the first liquid, the second liquid, and the emulsifier sequentially or simultaneously. For example, in particular embodiments, the dispersing agent is combined with the long chain polyunsaturated fatty acid source to form the blend in the second liquid and then the first liquid, the second liquid, and the emulsifier are combined to form the emulsion. For another example, in particular embodiments, the dispersing agent is combined with the LC-PUFA source and emulsifier to form the blend in the second liquid and then the first liquid and the second liquid are combined to form the emulsion.

In particular embodiments, the step of combining comprises mixing the emulsifier into the first liquid, mixing the dispersing agent with the polyunsaturated fatty acid source to form the blend in the second liquid, and then homogenizing the first liquid and the second liquid to form the emulsion. For example, the first liquid and the emulsifier may be used to form a mucilage or emulsifier solution. Then, the mucilage comprising the first liquid and the emulsifier may be combined with the second liquid, which includes the dispersing agent mixed with the polyunsaturated fatty acid, to form a pre-emulsion. The pre-emulsion can then be homogenized to form the emulsion.

Also, in some embodiments, the first liquid, the second liquid, and the emulsifier may be combined simultaneously by a homogenizing process. In yet other embodiments, the step of combining may comprise forming an emulsion concentrate comprising a portion of the first liquid, the second liquid and the emulsifier and then adding the remainder portion of the first liquid to the emulsion concentrate to form the emulsion comprising the discontinuous liquid phase and the continuous liquid phase.

The present disclosure also provides for a method of making an emulsion comprising providing a first liquid, providing a second liquid, and combining the first liquid, the second liquid, and an emulsifier so as to form an emulsion concentrate. Embodiments of the second liquid comprise a blend that includes a long chain polyunsaturated fatty acid source and a dispersing agent. The emulsion concentrate may be dosed into a third liquid where the emulsion concentrate disperses quickly to form an emulsion. In particular embodiments, homogenization of the emulsion concentrate with the third liquid is not required to form a stable emulsion. Embodiments of the first liquid and third liquid may comprise components of the continuous liquid phase as described above. In particular embodiments, the emulsion may comprise a beverage, a herbal composition, or a pharmaceutical composition, for example.

Embodiments of the present invention may also include emulsions which are spray dried, spray dried and coated, or spray dried and agglomerated. It should be understood by a person of ordinary skill in the art that embodiments of the emulsion may be spray dried by any method known in the art for spray drying. In addition, it should be understood by a person of ordinary skill in the art that embodiments of the spray dried emulsions may be coated or agglomerated with other components. For example, embodiments of the spray dried emulsions may be coated or agglomerated by sugars and maltodextrin or combinations thereof. Furthermore, understood by a person of ordinary skill in the art that embodiments of the spray dried emulsions may be coated or agglomerated by any method known in the art for coating or agglomerating. In particular embodiments, the spray dried, spray dried and coated, and/or spray dried and agglomerated emulsions can be added to liquid compositions wherein the emulsions disperse and form a liquid emulsion. For example, embodiments of spray dried, spray dried and coated, or spray dried and agglomerated may be added to a beverage to form a beverage emulsion.

The emulsions created by embodiments of the method of the present invention are stable and provide protection for the long chain polyunsaturated fatty acid from oxidation. In addition, the embodiments of the emulsions of the present invention may improve the bioavailability of the polyunsaturated fatty acids as compared to bulk oil.

Without being bound by theory, it is believed that in particular embodiments a sufficient amount of the dispersing agent reduces the viscosity and surface tension of the discontinuous liquid phase such that homogenization efficiency of the emulsion is improved. Thus, the viscosity difference between the continuous liquid phase and the discontinuous liquid phase plays a role in determining the effectiveness of the emulsification/homogenization processes. Since LC-PUFA sources, such as LC-PUFA oils, are hydrophobic and viscous, these properties can reduce the effectiveness of homogenization. Therefore, the dispersing agent is included in the discontinuous liquid phase to reduce the viscosity of the discontinuous liquid phase to a viscosity lower than the viscosity of the LC-PUFA source. Inclusion of the dispersing agent in the discontinuous liquid phase also results in the discontinuous liquid phase having a surface tension less than the surface tension of the LC-PUFA source. Consequently, the discontinuous liquid phase, which includes the LC-PUFA source, is more easily dispersed in the emulsion.

Moreover, having smaller discontinuous liquid phase particle size and higher continuous liquid phase viscosity can improve emulsion stability. Addition of the dispersing agent to the emulsion to form the blend having the LC-PUFA source also reduces the discontinous liquid phase particle size to improve homogenization efficiency.

Furthermore, Stokes' law indicates that the discontinuous liquid phase density affects emulsion's stability. In particular, Stokes' law indicates that emulsion stability can be enhanced by reducing the density difference between the continuous liquid phase and discontinuous liquid phase. By adding a dispersing agent to the discontinuous liquid phase, the discontinuous liquid phase density can be adjusted. In some embodiments, the LC-PUFA has first vicosity and the dispersing agent has a second viscosity less than the first density. Thus, in particular embodiments, a higher percentage of the dispersing agent in the discontinuous liquid phase results in a lower discontinuous liquid phase density. For example, in embodiments where substantially all of the discontinuous liquid phase comprises a second liquid having a blend of a dispersing agent and a LC-PUFA oil, the discontinuous liquid phase density decreases as the percentage of dispersing agent in the blend increases (i.e., the percentage of LC-PUFA oil percentage decreases).

An additional benefit of producing a stable emulsion comprising the LC-PUFA source is that at least a portion of any degradation or oxidation of the LC-PUFA is inhibited, suppressed, or reduced by forming the emulsion having the long chain polyunsaturated fatty acid source. In particular embodiments, substantially all of the degradation of the long chain polyunsaturated fatty acid is inhibited, suppressed, or reduced by forming the emulsion having the long chain polyunsaturated acid source. Thus, without being bound by theory, it is believed that in embodiments where the LC-PUFA has an odor or taste, forming the emulsion having the at least one the long chain polyunsaturated acid substantially masks the odor or the taste of the at least one long chain polyunsaturated fatty acid source (e.g., a fishy odor). Blending the dispersing agent with the LC-PUFA source also reduces oxidation of the LC-PUFA source. For example, it is believed that dispersing agents which are more polar form a protective layer between the LC-PUFA source and the continuous liquid phase. Also, it is believed that antioxidant dispersing agents such as vitamin E, ascorbyl palmitate, and rosemary extract help to protect the LC-PUFA from oxidation. Furthermore, the reduction of degradation of the LC-PUFA increases the emulsion shelf-life.

In use, embodiments of emulsions of the present invention may be functional in compositions such as beverages, herbal compositions, pharmaceutical compositions, or the like, which may be ingested or otherwise introduced in to a consumer such that the LC-PUFA, and its beneficial properties can be benefited from. In embodiments wherein the emulsion are ingested, the present invention provide for an emulsion wherein the LC-PUFA is substantially undetectable to the taste or smell of the consumer. Thus, an emulsion can be ingested by a consumer without any undesirable odor, taste, or like property.

Other embodiments are further illustrated below in the examples which are not to be construed in any way as imposing limitations upon the scope of this disclosure. On the contrary, it is to be clearly understood that resort may be had to various other embodiments, modifications, and equivalents thereof which, after reading the description therein, may suggest themselves to those skilled in the art without departing from the scope of this disclosure and the appended claims.

### EXAMPLE 1

### Effect of Oil Additive on Mean Particle Size Distribution of DHA Emulsions

Two 250 g emulsions were produced using the formulation of Tables 2 and 3 below, where MCT is medium chain triglyceride and OD is orange distillate:

TABLE 2

| Trial run # | |
|---|---|
| 1 | 2 |
| Emulsifier used | |
| Ticamulsion | Ticamulsion |
| MCT | (MCT/OD) |

Unit

| | wt % | gram | wt % | gram |
|---|---|---|---|---|
| Ticamulsion | 17.5 | 43.75 | 17.5 | 43.75 |
| Martek DHA oil | 11.25 | 28.13 | 11.25 | 28.13 |
| MCT | 3.75 | 9.37 | 1.875 | 4.685 |
| OD | 0 | 0 | 1.875 | 4.685 |
| Sodium Benzoate | 0.13 | 0.33 | 0.13 | 0.33 |
| Citric Acid | 0.20 | 0.50 | 0.20 | 0.50 |
| Processed Water | 67.17 | 167.92 | 67.17 | 167.92 |
| Total | 100 | 250 | 100 | 250 |

TABLE 3

| Trial run # | |
|---|---|
| 3 | 4 |
| Emulsifier used | |
| Ticamulsion | Ticamulsion |
| 5X folded | Orange |
| lime oil | Distillate |

Unit

| | wt % | gram | wt % | gram |
|---|---|---|---|---|
| Ticamulsion | 17.5 | 43.75 | 17.5 | 43.75 |
| Martek DHA oil | 11.25 | 28.13 | 11.25 | 28.13 |
| 5X Folded Lime | 3.75 | 9.37 | 0 | 0 |
| oil | | | | |
| Orange distillate | 0 | 0 | 3.75 | 9.37 |
| Sodium Benzoate | 0.13 | 0.33 | 0.13 | 0.33 |
| Citric Acid | 0.20 | 0.50 | 0.20 | 0.50 |
| Processed Water | 67.17 | 167.92 | 67.17 | 167.92 |
| Total | 100 | 250 | 100 | 250 |

The emulsions were prepared by first preparing a mucilage by weighting water content for the batch in a 600 ml beaker. The beaker was placed under a propeller based agitator. Sodium benzoate was added to the mixing vortex and the emulsifier solution was mixed for 3 minutes. Citric acid was added to the mixing vortex and the emulsifier solution was mixed for 3 minutes. Emulsifier was slowly added to the mixing vortex and agitation was continued for 1 hour. The emulsifier solution was placed on a table overnight to allow foam to separate.

A pre-emulsion was prepared by placing the filtered emulsifier solution (the mucilage was through a 100 mesh screen) under a propeller based agitator and oil blend of DHA oil and orange distillate, medium chain triglyceride, and/or folded oil was slowly added to the mixing vortex to produce a coarse emulsion. The coarse emulsion solution was transferred to the high shear mixer (Polytron PT3100 or Pri Sci 250). The mixer speed was set at 4 and the emulsifier solution was mixed for 2 minutes to yield a pre-emulsion.

The emulsion was prepared by running DI water through a NanoMizer and adjust the plunger speed to achieve homogenization pressure of 31 MPa (4500 psi). The pre-emulsion was homogenized twice at the desired homogenization pressure. If necessary, the plunger speed was adjusted to achieve the desired homogenization pressure. The emulsion was then packaged and stored in chilled conditions. Tables 4 and 5 summarizes the results of a particle size measurement of the emulsion and shows that addition of orange distillate, medium chain triglycerides and/or folded oils to DHA oils can significantly improve ease of emulsification of DHA oils.

TABLE 4

| | | Particle Size (μm) |
|---|---|---|
| Trial run # | Description | Mean |
| 1 | DHA oils with MCT | 0.277 |
| 2 | DHA oils with MCT/OD | 0.207 |

TABLE 5

| | | Particle Size (μm) |
|---|---|---|
| Trial run # | Description | Mean |
| 3 | DHA oils with with 5X Folded Lime | 0.151 |
| | Oil | |
| 4 | DHA oils with Orange Distillate | 0.154 |

### EXAMPLE 2

Omega-3 fatty acid oil-in-water emulsions with 17.5% Eficacia or 17.5% Ticamulsion 2010A, dl-limonene, and 15% Martek DHA oil were prepared using the procedures of EXAMPLE 1. The omega-3 fatty acid oil was supplied by Martek Bioscience and was stabilized with an antioxidant mixture system of tocopherols, ascorbyl palmitate, soy lecithin and rosemary extract. Eficacia, a special grade of gum arabic, was provided by CNI. Ticamulsion 2010A, a modified gum aracia, was supplied by TIC Gum. All the components were used without further purification.

Omnion's Food Stability Analyzer (FSA) was been employed to determine antioxidant efficacy in retarding lipid oxidation of the omega-3 fatty emulsions. FSA conducted accelerated oxidation studies using the combination of elevated temperature (up to 150° C.) and catalysis, a proprietary heavy metal complex. The degree of oxidation acceleration for the combination was on an order of several hundred times faster than the real shelf life study. The FSA instrument measured oxygen concentration at the headspace of the sample cell (40 c.c. in volume or ˜2×10²⁰ oxygen molecules). The FSA method was significantly more sensitive than the conventional oxygen bomb method in which high pressure pure oxygen and elevated temperature. The end point determination of the FSA for the oxidative stability analysis was determined by the inflection point (sharp slope change) of the oxygen concentration versus time curve where the added antioxidants are consumed and the lipid auto-oxidation starts to accelerate. In addition, the end point typically indicated the time that it takes 5% of headspace oxygen or ˜10¹⁹ oxygen molecules to be consumed by the substrate studied.

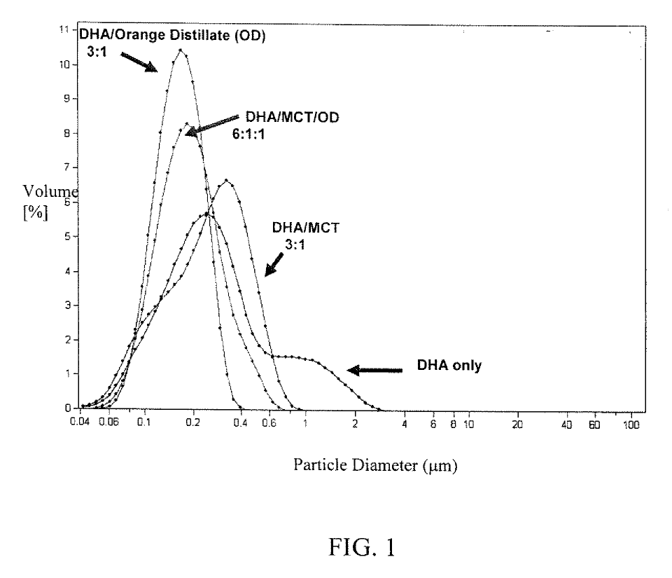

The Saffest® system was used to analyze the oxidative degradation of in-house Omega-3 emulsions. The Saffest® system is a calorimetric method (based on the ferric thiocyanate method modified for safety reasons by replacing benzene:methanol with isopropanal) and is AOAC certified. The SafTest® system provided a rapid determination of peroxide values of the emulsion studied and the results are summarized in Tables 6 and 7 and FIG. 1. Thus, the result indicated that addition of the oils and polyphenols to the DHA emulsions reduced oxidative degradation.

TABLE 6
Effect of addition of orange distillate and
MCT on the oxidative stability

| | Concentration | End Point | End Point | End Point |
|---|---|---|---|---|
| Source | (Active) | (hour) | (hour) | (hour) |
| Oil Blend | | DHA oil | DHA:Orange | DHA:MCT |
| Composition | | only | Distillate | 3:1 ratio |
| | | | 3:1 ratio | |
| Chinese | 0 ppm | 1.3 | 1.4 | 1.8 |
| Green | 1000 ppm | 21.4 | 34 | 34 |
| Tea | 2000 ppm | 27.3 | 48 | 54 |
| Polyphenols | 3000 ppm | 35 | 99 | 78 |
| T40 | 4000 ppm | 51.5 | ˜115 | ˜103 |

TABLE 7
Peroxide value (PV) of Aged In-house Omega-3 emulsions

| | | Initial | | |
|---|---|---|---|---|
| | | Mean | | |
| | | Particle | Mean Particle | |
| Emulsifier/Oil | | Size (μm) | Size (μm) after | Peroxide |
| | Oil Blend | Time = | 26 weeks of | Value |
| Emulsifier | Composition | 0 week | storage at 4° C. | (MEQ/Kg) |
| 10% | 20% DHA | 0.337 | 0.522 | 65 |
| Ticamulsion | only | | | |
| 10% | 20% Oil Blend | 0.246 | 0.409 | 28.3 |
| Ticamulsion | 3:1 | | | |
| | DHA:dl-limonene | | | |
| 10% | 20% Oil Blend | 0.218 | 0.399 | 21.6 |
| Ticamulsion | 2:1 | | | |
| | DHA:dl-limonene | | | |
| 10% | 20% Oil Blend | 0.197 | 0.394 | 9.5 |
| Ticamulsion | 1:1 | | | |
| | DHA:dl-limonene | | | |

Green tea polyphenols (GTP) were used to examine the antioxidant efficacy of GTP in retarding off taste development due to Omega-3 oil oxidation in embodiments of the emulsion. Table 8 shows the ongoing shelf stability evaluation of Fanta Orange Zero fortified with DHA oil and different additives. The results indicate that after 12 weeks of ambient storage the test samples with GTP have not developed fishy smell and taste. However, the test sample with vitamin C only did develop fishy taste and smell after 3 weeks of ambient storage. In addition, sensory results of 100 people consumer study show after 12 weeks of ambient storage Fanta Zero Orange fortified with DHA oil and vitamin C/EDTA develops slight fishy smell and taste although the off taste can be masked by the use of cooling agents. Hence, green tea polyphenols should be able to replace vitamin C and EDTA in Omega-3 emulsions and Omega-3 fortified beverages to retard lipid oxidation and ensure shelf stability.

TABLE 8
Sensory Evaluation of Fanta Orange Zero with different additives

| | Test Formula | Test | |
|---|---|---|---|
| Beverage | Per serving amount | Condition* | Comment |
| Fanta | 32 mg DHA per | RT | After 12 weeks, there is no fishy taste |
| Orange | serving | | and smell developed |
| Zero | 25 ppm of Chinese | | |
| | green tea polyphenols | | |
| Fanta | 32 mg DHA per | RT | After 12 weeks, there is no fishy taste |
| Orange | serving | | and smell developed |
| Zero | 50 ppm of Chinese | | |
| | green tea polyphenols | | |
| Fanta | 32 mg DHA per | RT | After 12 weeks, there is no fishy taste |
| Orange | serving | | and smell developed |
| Zero | 75 ppm of Chinese | | Slight bitter after taste |
| | green tea polyphenols | | |
| Fanta | 32 mg DHA per | RT | After 12 weeks, the results of 100 |
| Orange | serving 100% | | people consumer taste test indicate |
| Zero | Vitamin C per | | there is development of slight fishy |
| | serving + 20 ppm | | smell and taste, however, the slight |
| | EDTA | | off taste can be masked by low use |
| | | | level of cooling agents |
| Fanta | 32 mg DHA per | RT | After 12 weeks, the results of 100 |
| Orange | serving 100% | | people consumer taste test indicate |
| Zero | Vitamin C per | | there is no fishy smell and taste |
| | serving + 20 ppm | | |
| | EDTA + 10 ppm | | |
| | WS-3/WS- | | |
| | 23 cooling agent | | |
| Fanta | 32 mg DHA per | RT | After 12 weeks, the results of 100 |
| Orange | serving 100% | | people consumer taste test indicate |
| Zero | Vitamin C per | | there is no fishy smell and taste |
| | serving + 20 ppm | | |
| | EDTA + 125 ppm | | |
| | IFF | | |
| | SN451138 cooling | | |
| | agent | | |
| Fanta | 32 mg DHA per | RT | After 3 weeks, the sample tasted and |
| Orange | serving using Nanoemulsion | | smelled fishy |
| Zero | with 100% | | |
| | Vitamin C per | | |
| | serving | | |

RT: Room Temperature (˜23° C.)

### EXAMPLE 3

A 200 kg omega-3 fatty acid oil-in-water stable emulsion was made according to the formulation of Table 9 using the procedures of EXAMPLE 1.

TABLE 9

Unit

| | wt % | kg |
|---|---|---|
| Ticamulsion | 17.5 | 35 |
| Martek DHA oils | 15 | 30 |
| CP Orange Oils | 5 | 10 |
| Sodium Benzoate | 0.13 | 0.26 |
| Citric Acid | 0.2 | 0.4 |
| Green Tea Extract | 0.3 | 0.6 |
| EDTA | 0.1 | 0.2 |
| Processed Water | 61.77 | 123.54 |
| Total | 100 | 200 |

### EXAMPLE 4

An omega-3 fatty acid oil-in-water emulsion with 17.5% Ticamulsion 2010A was prepared using the formula in Table 10 below. Sucrose diacetate hexa-isobutyrate was used as the weight agent.

TABLE 10

| Component | wt % | grams |
|---|---|---|
| Ticamulsion | 17.5 | 175 |
| Martek DHA oil | 15 | 150 |
| Sucrose diacetate | 2.5 | 25 |
| hexa-isobutyrate | | |
| Orange distillate | 2.5 | 25 |
| Sodium Benzoate | 0.13 | 1.3 |
| Citric Acid | 0.20 | 2 |
| Processed Water | 62.17 | 621.7 |
| Total | 100 | 1000 |

The emulsion was prepared by first preparing a mucilage by weighting water content for the batch in a 2000 ml beaker. The beaker was placed under a propeller based agitator. Sodium benzoate was added to the mixing vortex and the emulsifier solution was mixed for 3 minutes. Citric acid was added to the mixing vortex and the emulsifier solution was mixed for 3 minutes. Emulsifier was slowly added to the mixing vortex and agitation was continued for 1 hour. The emulsifier solution was placed on a table overnight to allow foam to separate.

A pre-emulsion was prepared by placing the emulsifier solution under a propeller based agitator and oil blend of DHA oil, sucrose diacetate hexa-isobutyrate and orange distillate, medium chain triglyceride, and/or folded oil was slowly added to the mixing vortex to produce a coarse emulsion. The coarse emulsion solution was transferred to the high shear mixer, Polytron PT3100. The mixer speed was set at 410,000 rpm and the emulsifier solution was mixed for 2 minutes to yield a pre-emulsion.

The emulsion was prepared by running DI water through an APV 1000 homogenizer and adjust the homogenization pressure of 4500 psi. The pre-emulsion was homogenized twice at the desired homogenization pressure. If necessary, the plunger speed was adjusted to achieve the desired homogenization pressure. The emulsion was then packaged and stored in chilled conditions. The resulting emulsion had a discontinuous liquid phase with a mean particle size of 0.173 μm.

It should be understood that the foregoing relates to particular embodiments of the present invention, and that numerous changes may be made therein without departing from the scope of the invention as defined from the following claims.

We claim:

1. An emulsion comprising:
a continuous liquid phase;
an emulsifier; and
a discontinuous liquid phase comprising a blend including a polyunsaturated fatty acid source and a dispersing agent, the polyunsaturated fatty acid source comprising at least one polyunsaturated fatty acid, wherein the weight ratio of the fatty acid source to the dispersing agent in the blend ranges from about between 9:1 and 1:10.

2. The emulsion of claim 1, wherein the weight ratio of the fatty acid source to the dispersing agent in the blend ranges from about between 5:1 and 1:1.

3. The emulsion of claim 1, wherein the weight ratio of the fatty acid source to the dispersing agent in the blend ranges from about between 4:1 and 3:1.

4. The emulsion of claim 1, wherein the emulsion is an emulsion concentrate.

5. The emulsion of claim 1, wherein the emulsion is contained in a beverages a herbal composition, a pharmaceutical composition or combinations thereof

6. The emulsion of claim 1, wherein the dispersing agent is selected from the group of vitamin E, ascorbyl palmitate, rosemary extract, a terpene, a flavor oil, vegetable oil, an essential oil, or combinations thereof.

7. The emulsion of claim 6, wherein the dispersing agent comprises a terpene selected from the group of d-limonene, 1-limonene, dl-limonene, orange distillate oil, and combinations thereof.

8. The emulsion of claim 1, wherein the emulsifier comprises a carbohydrate-based macromolecule.

9. The emulsion of claim 8, wherein the carbohydrate-based macromolecule is selected from the group of gum acacia, modified food starch, gum ghatti, pectin, beta-pectin, modified gum acacia, and combinations thereof.

10. The emulsion of claim 1, wherein the continuous liquid phase has a pH between 2 and 7.

11. The emulsion of claim 1, wherein the discontinuous liquid phase further comprises at least one folded oil selected from the group of 4-fold bergamot oil, bergaptene free bergamot oil, terpeneless grapefruit oil, 4-fold grapefruit oil, 5-fold grapefruit oil, 6-fold grapefruit oil, 10-fold grapefruit oil, high aldehyde grapefruit oil, 5-fold grapefruit juice extract, 7-fold grapefruit juice extract, terpencless lemon oil, 2-fold lemon oil, 3-fold lemon oil, 5-fold lemon oil, 10-fold lemon oil, 13-fold lemon oil, washed 5-fold lemon oil, 10-fold lemon oil, Sesquiterpeneless lemon oil, FC free lemon oil, distilled 3-fold lime oil, distilled 4-fold lime oil, distilled 5-fold lime oil, distilled terpeneless lime oil, distilled sesquiterpeneless lime oil, distilled washed 5 fold lime oil, cold pressed 3-fold lime oil, cold pressed 4-fold lime oil, cold pressed 5-fold lime oil, cold pressed 10-fold lime oil, cold pressed terpeneless lime oil, 4-fold mandarin oil, 5-fold mandarin oil, 10-fold mandarin oil, terpeneless orange oil, 2-fold orange oil, 3-fold orange oil, 4-fold orange oil, 5-fold orange oil, 7-fold orange oil, 8-fold orange oil, 10-fold orange oil, 15-fold orange oil, 20-fold orange oil, 25-fold orange oil, 30-fold orange oil, 5-fold orange juice extract, 8-fold orange juice extract, 3-fold tangerine oil, 5-fold tangerine oil, terpeneless tangerine oil, and combinations thereof.

12. The emulsion of claim 1, wherein the discontinuous liquid phase further comprises a medium chain triglyceride.

13. The emulsion of claim 1, wherein the continuous liquid phase further comprises at least one polyphenol.

14. The emulsion of claim 13, wherein the at least one polyphenol comprises green tea extract.

15. The emulsion of claim 1, wherein the polyunsaturated fatty acid source comprises an omega-3 fatty acid oil selected from the group of alpha-linolenic acid oil, eicosapentaenoic acid oil, docosahexaenoic acid oil, and combinations thereof

16. A method for making an emulsion comprising:
providing a first liquid,
providing a second liquid comprising a blend including a long chain polyunsaturated fatty acid source and a dispersing agent, wherein the weight ratio of the polyunsaturated fatty acid source to the dispersing agent in the blend ranges from about between 9:1 and 1:10, the long chain polyunsaturated fatty acid source comprising at least one long chain polyunsaturated fatty acid; and
combining the first liquid, the second liquid, and an emulsifier so as to form the emulsion comprising a continuous liquid phase including the first liquid and a discontinuous liquid phase including the second liquid.

17. The method of claim 16, wherein the weight ratio of the long chain polyunsaturated fatty acid source to the dispersing agent in the blend ranges from about 5:1 to about 1:1.

18. The method of claim 16, wherein the weight ratio of the long chain polyunsaturated fatty acid source to the dispersing agent in the blend ranges from about 4:1 to about 3:1.

19. A method for inhibiting, suppressing, or reducing at least a portion of any degradation of at least one omega-3 fatty acid in a composition, the method comprising providing at least one polyphenol in the composition.

20. The method of claim 1, wherein substantially all of the degradation of the omega-3 fatty acid is inhibited, suppressed, or reduced by providing the at least one polyphenol in the composition.

21. An emulsion comprising:
a continuous liquid phase;
an emulsifier; and
a discontinuous liquid phase comprising a blend including a polyunsaturated fatty acid source and a folded oil, the polyunsaturated fatty acid source comprising at least one polyunsaturated fatty acid.

22. An emulsion comprising:
a continuous liquid phase;
an emulsifier; and
a discontinuous liquid phase comprising a blend including a polyunsaturated fatty acid source, a weighing agent, and a dispersing agent, the polyunsaturated fatty acid source comprising at least one polyunsaturated fatty acid.

23. The emulsion of claim 22, wherein the weighing agent is selected from the group of a brominated vegetable oil, an ester gum, sucrose diacetate hexa-isoburtyurate, a refined gum dammar, a ganuaba wax, benzyl benzoate, a polyglyceryl ester, a glyceryl tribenzoate, and combinations thereof.

24. The emulsion of claim 22, wherein the continuous liquid phase further comprises a sugar.

25. A method for improving bioavailability of a polyunsaturated fatty acid comprising:
providing an emulsion comprising:
a continuous liquid phase;
an emulsifier; and
a discontinuous liquid phase comprising a blend including a polyunsaturated fatty acid source and a dispersing agent, the polyunsaturated fatty acid source comprising the polyunsaturated fatty acid, the polyunsaturated fatty acid source comprising at least one polyunsaturated fatty acid, wherein the weight ratio of the fatty acid source to the dispersing agent in the blend ranges from about between 9:1 and 1:10.

26. A method for administering a polyunsaturated fatty acid comprising:
providing an emulsion comprising:
a continuous liquid phase;
an emulsifier; and
a discontinuous liquid phase comprising a blend including a polyunsaturated fatty acid source and a dispersing agent, the polyunsaturated fatty acid source comprising the polyunsaturated fatty acid, the polyunsaturated fatty acid source comprising at least one polyunsaturated fatty acid, wherein the weight ratio of the fatty acid source to the dispersing agent in the blend ranges from about between 9:1 and 1:10.

27. The emulsion of claim 1, wherein the emulsion further comprises β-carotene, enzymes, carotenoids, eucalyptol, eugerol, gingerol, avenacoside, phenolic acids, flavonoids, coumarins, proanthocyanidins, curcuminoids, Vitamin E, Vitamin K, or combinations thereof.

28. The emulsion of claim 1, wherein the continuous liquid phase further comprises a water dispersible bioactive comprising lutein.

29. The emulsion of claim 1, wherein the discontinuous liquid phase further comprises an oil dispersible bioactive comprising lutein.

30. The emulsion of claim 1, wherein the continuous liquid phase further comprises a water dispersible bioactive comprising lutein and a polyphenol.

31. The emulsion of claim 1, wherein the discontinuous liquid phase further comprises an oil dispersible bioactive comprising lutein and the continuous phase further comprises a polyphenol.
